(19) United States
(12) Patent Application Publication
(10) Pub. No.: US 2010/0204706 A1
(43) Pub. Date: Aug. 12, 2010

(54) Intracorneal Lens Insertion Device

(76) Inventors: Vladimir Feingold, Laguna Niguel, CA (US); Alexei Kosmynine, Aliso Viejo, CA (US)

Correspondence Address: SHOEMAKER AND MATTARE, LTD, 10 POST OFFICE ROAD - SUITE 100, SILVER SPRING, MD, 20910, US

(21) Appl. No.: 12/766,295

(22) Filed: Apr. 23, 2010

Related U.S. Application Data
(62) Division of application No. 12/167,950, filed on Jul. 3, 2008.
Application No. 12/167,950 is a continuation of application No. 10/883,465, filed on Jul. 1, 2004, now abandoned.

(51) Int. Cl. A61F 9/007

(52) U.S. Cl. 606/107

(57) ABSTRACT

An opthalmologic lens insertion device holds a lens between a pair of leaf springs and has a plunger that moves between the leaf springs to eject the lens onto the cornea or into a corneal pocket.

### RELATED APPLICATIONS

This application is division of application Ser. No. 12/167,950, filed Jul. 3, 2008, which was a continuation of application Ser. No. 10/883,465 filed Jul. 1, 2004, now abandoned, the content of which is incorporated by reference herein.

### FIELD OF THE INVENTION

The invention relates to instruments for inserting a lens into the cornea.

### BACKGROUND

Insertion of a lens into the cornea requires precise and careful placement. There is a need for an instrument that is easy to use, will not fold or damage the lens, and will fit into a corneal pocket.

### SUMMARY OF THE INVENTION

This invention is embodied in a device that has a pair of gripping members that extend from a handle to a cotermination point where they can grip a lens. A plunger is moveable between the gripping members from a retracted position to an extended position whereby it will push the lens out from between the gripping members. The gripping members are resiliently biased so that while they grip the lens sufficiently to hold it, when the plunger passes between them at the cotermination point they will separate and allow the plunger to engage the lens and push it out of the cotermination point and into the desired position in the cornea.

The device has a handle and the gripping members are attached to the handle, preferably separated. The plunger is held inside the handle in a slide mechanism that allows it to slide back and forth. It can be actuated by an actuator that is hand operable. The actuator is outside the handle and is attached to the plunger by way of a slot in the handle.

### BRIEF DESCRIPTION OF THE DRAWINGS

FIG. 1 is a perspective view of the invention.

FIG. 1A is a perspective view of the invention indicating a portion enlarged at FIG. 9.

FIG. 2 is a top view of the invention.

FIG. 3 is a detail view of the cotermination point.

FIG. 4 is a view through section 4-4 of FIG. 2.

FIG. 5 is a bottom view of the invention.

FIG. 6 is a view through 6-6 of FIG. 5.

FIG. 7 is side view of the invention.

FIG. 8 is a view through section 8-8 of FIG. 7.

FIG. 9 is a partial perspective view of the front of the invention.

FIG. 10 is a partial perspective and sectioned view of the invention with the plunger in a retracted position.

FIG. 11 is a partial perspective top section view of the invention with the plunger in a retracted position.

FIG. 12 is a partial perspective side section view of the invention showing the plunger in a retracted position and a lens at the cotermination point of the gripping members.

FIG. 13 is a partial perspective view of the invention with the plunger in the extended position and a lens having been pushed out from the cotermination point of the gripping members.

FIG. 14 is a partial perspective view of the invention with the gripping members shown in transparency and the plunger in a retracted position and a lens in the cotermination point.

FIG. 15 is a partial perspective top section view of the invention showing the plunger in a retracted position.

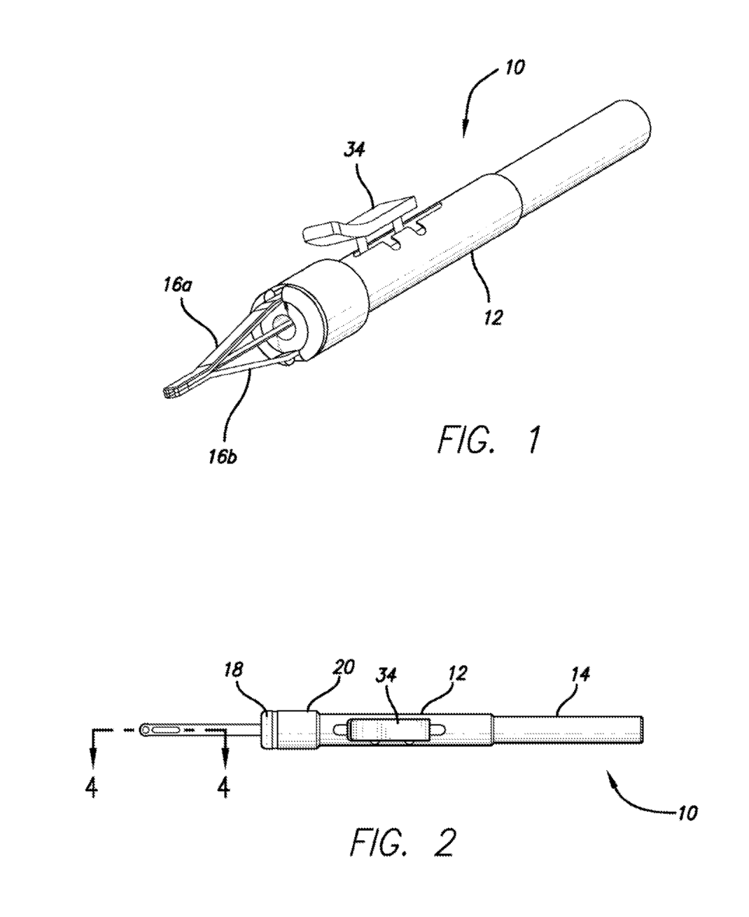

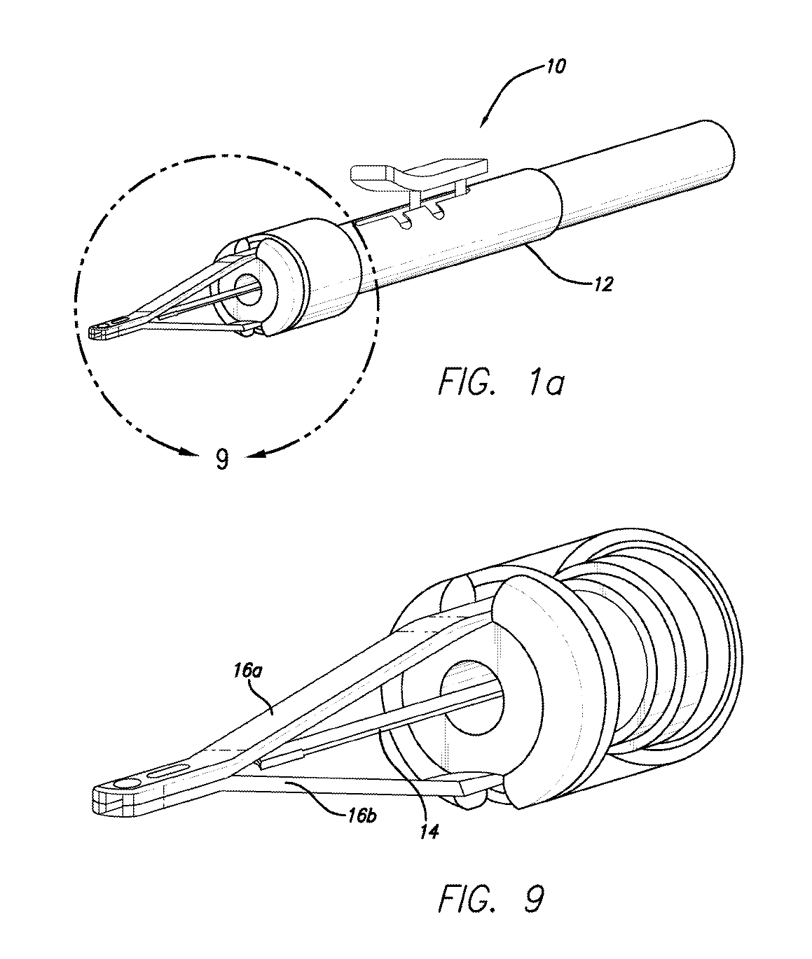

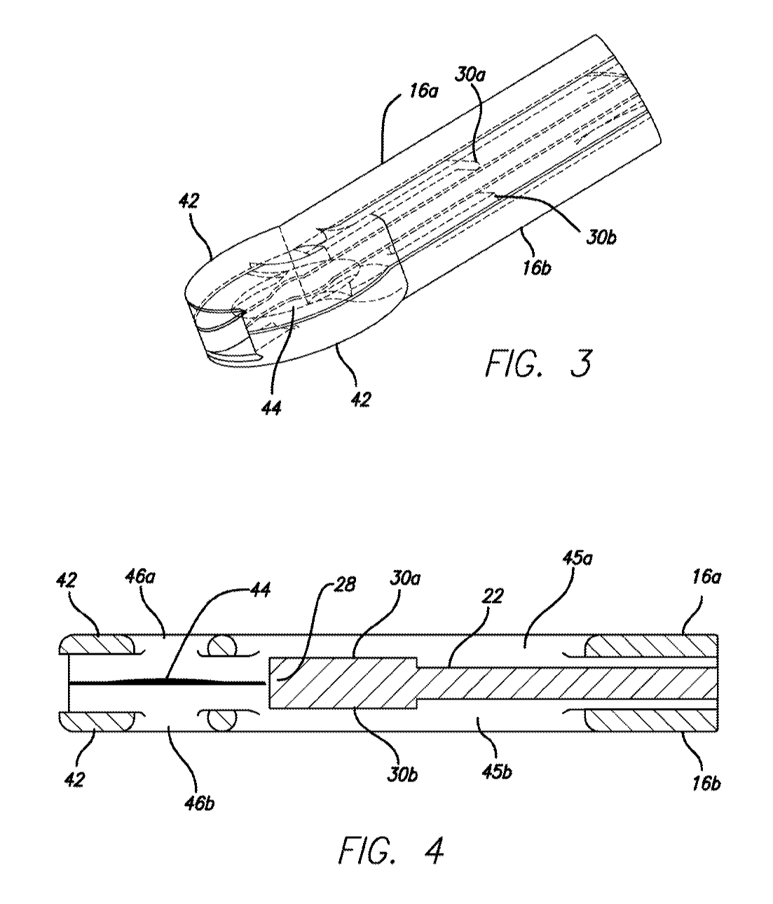

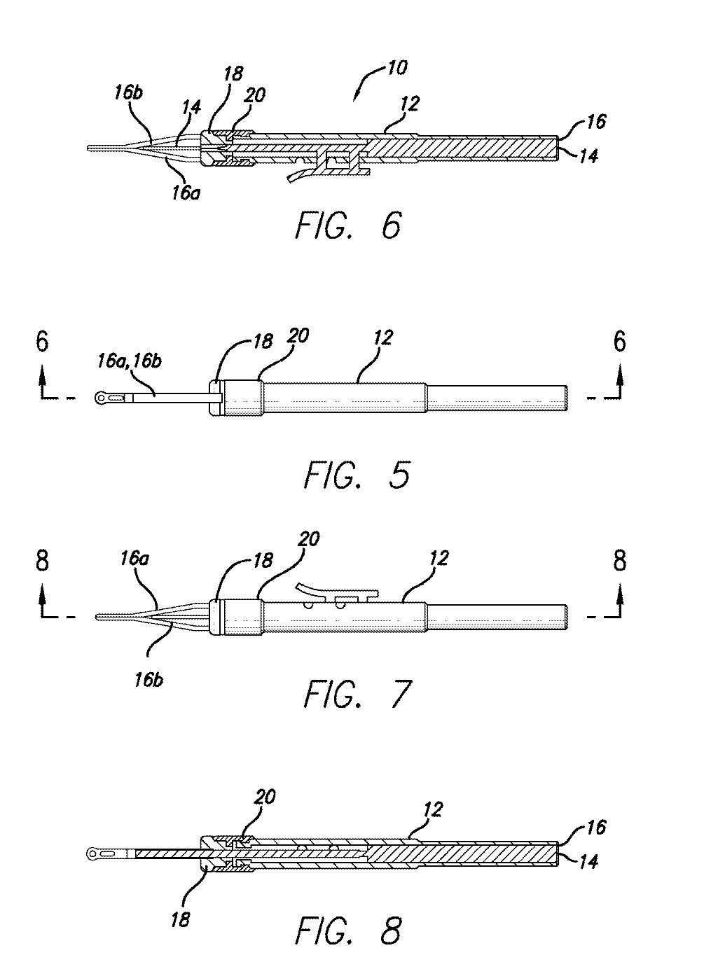

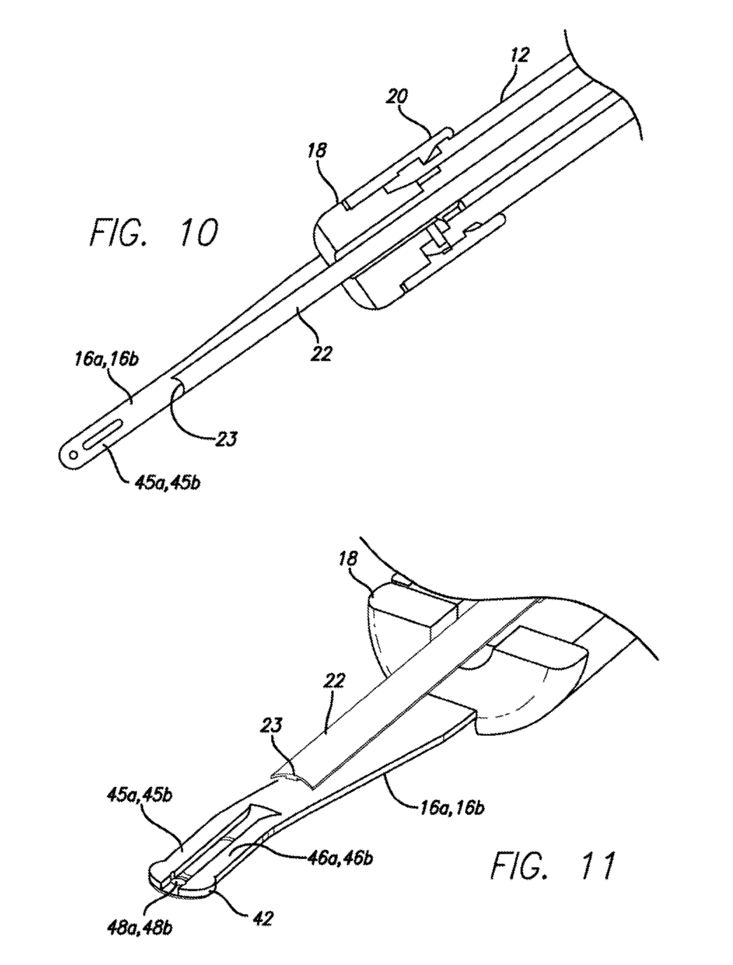

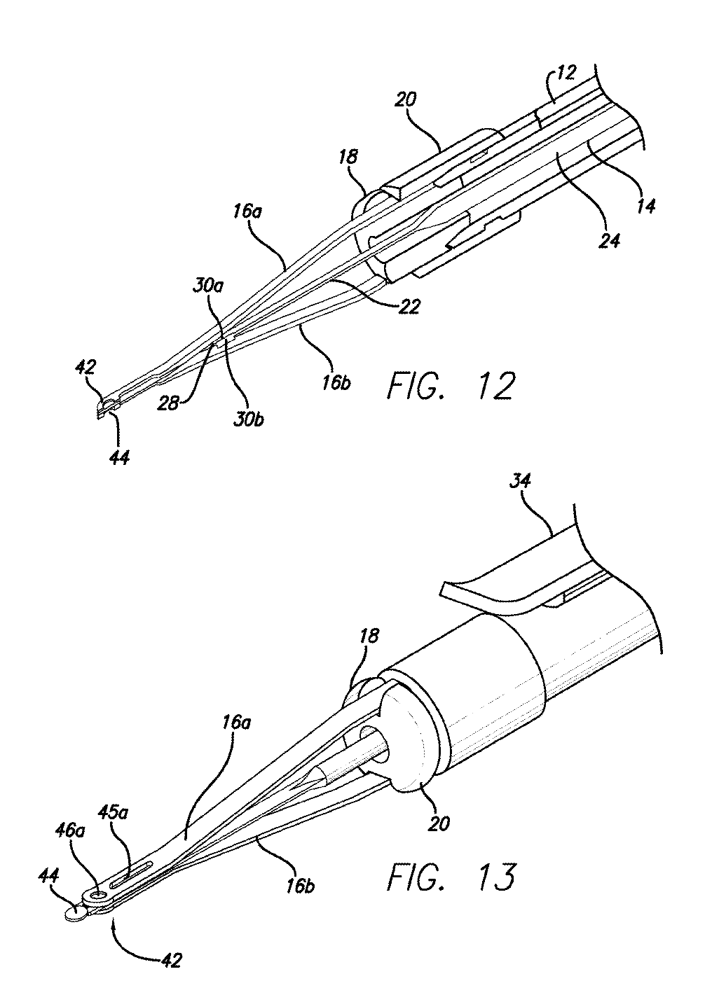

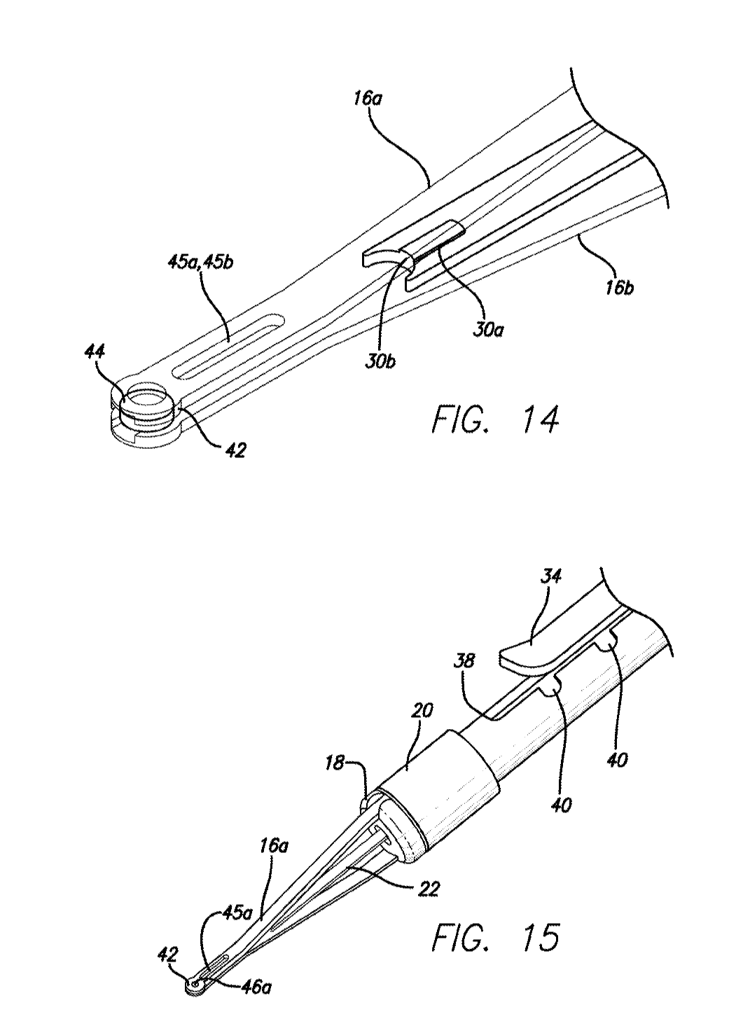

### DESCRIPTION OF PREFERRED EMBODIMENTS

The present invention is a device that is held in the hand of a surgeon performing a lens insertion procedure to place a lens into a human or other animal eye. Typically a lens is inserted under a corneal flap or into a corneal pocket. This device allows insertion in particular into a corneal pocket or on a corneal surface exposed by a flap and controllable deposit of the lens in the correct place.

The following description can be best understood with reference to the figures and the reference numerals assigned to the parts of the device.

The device 10 has three major portions. These are, a handle 12 a plunger 14 and grippers 16a and 16b. At the front of the handle are a bearing 18 and a retaining collar 20.

The plunger 14 has front portion 22 and a shaft portion 24 and control portion 26. The front portion 22 of the plunger 14 has a curved tip 28 and oppositely thickened portions 30a and 30b that will be referred to as lands 30a and 30b. The front portion of the plunger 22 terminates in a curved termination end 23 configured to contact the curved periphery of the lens to be pushed out of the device. Behind the front portion 22 of the plunger 14, the plunger shaft portion 24 rides in the bearing 18. At the back of the plunger shaft portion 18, extending rearwardly is the plunger control portion 26 of the plunger 14. It rides smoothly on the inside surface 32 of the handle 12 which serves as a bearing. Also extending laterally from the plunger 14 is an actuator 34, attached by posts 36 extending through the handle 12 by means of a slot 38. The slot 38 has radial slots 40.

The grippers 16a and 16b are fitted and held by the collar 20. They are mounted diametrically oppositely on the handle 12 so as to be spaced apart where they are attached to the handle 12. They are shaped to converge to meet at a cotermination point 42. The grippers 16a and 16b are preferably made of a spring material so that they can be resiliently biased together where they meet at the cotermination point 42. The cotermination point 42 is configured to hold a lens 44, and the grippers are sufficiently biased together to maintain the lens 44 in place until it is pushed out by the plunger 14. The grippers 16a and 16b also have recesses 45a and 45b on their inside surfaces starting just back of the cotermination point 42 and extending to the end of the grippers 16a and 16b. These recesses are also called grooves 45a and 45b in consideration of their interaction with the lands 30a and 30b. The grippers 16a and 16b also have windows 46a and 46b opening just behind the cotermination points 42 and extending rearwardly. There are also circular windows 48a and 48b in the cotermination points 42.

Use of the device commences with placing a lens 44 in the cotermination point 42. This is done by spreading the grippers 16a and 16b. The grippers 16a and 16b can be conveniently spread by rotating the actuator 34 so that its posts 36 enter the radial slots 40. This rotation will rotate the front portion 22 of the plunger 14 and as it rotates it will contact the grippers 16a and 16b and spread them apart, and hold them apart so that the lens 44 can be easily and accurately put in place.

The device is ready to use to put the lens 44 in place in a corneal pocket or on the cornea to be covered by a flap.

To insert the lens 44, for example, into a corneal pocket, the grippers 16a and 16b are slid into the corneal pocket, stopping just short of the place where the lens 44 will be placed. Then the plunger 14 is moved forward. As it moves forward the lands 30a and 30b enter the grooves 45a and 45b. This interaction has the benefit that the termination end 23 of the plunger 14 cannot override the lens 44. Also if the cooperative dimensioning of their lands 30a and 30b and the grooves 45a and 45b are made that the grooves 45a and 45b are not as deep as the lands 30a and 30b are high, by a small amount, the entry of the lands 30a and 30b into the grooves 45a and 45b will cause the grippers 16a and 16b to separate slightly releasing their grip on the lens 44. The operator can see the movement of the plunger termination end 23 through the windows 45a and 45b and of both it and the lens 44 through the windows 46a and 46b so that the insertion process can be carefully controlled. After the lens 44 has been ejected from the cotermination point 42 small adjustments may be made as needed by nudging the lens 44 with the cotermination points 42.

As is apparent, the lens is held sufficiently firmly between the grippers until the time for its ejection by the plunger. The plunger, in its retracted position is not in contact with the grippers, so only the grippers determine the holding of the lens prior to operating the device. The plunger has sufficient travel that it will move through the lens ejection cycle to pass slightly beyond the end of the grippers so that the lens is fully ejected.

The injector as described above can be used with a single hand and is suitable for introduction of a lens into a sub-3 mm corneal tunnel incision, and can be autoclaved.

Exemplary embodiments of the invention are disclosed herein. Thus it will be appreciated that various modifications, alternatives, variations, etc. may be made without departing from the spirit and scope of the invention as defined in the appended claims and equivalents.

What is claimed is:

1. An opthalmologic lens insertion device comprising:
a handle;
a pair of gripping members extending from the handle to a cotermination point configured to accept and grip a lens, the gripping members being resiliently biased toward each other to exert sufficient pressure at the cotermination point upon a lens in place between them to retain the lens in place;
a plunger extending from the handle between the gripping members and being moveable between a retracted position and an extended position, the plunger having a lens pushing end and the retracted position being sufficient to place the lens pushing end away from the cotermination point toward the handle and the extended position being sufficient to pass the lens pushing end between the gripping members at the cotermination point away from the handle such that a lens held in the cotermination point will be pushed out of the device by the movement of the lens pushing end.

2. The device of claim 1, wherein the plunger is fitted inside the handle for reciprocal movement between the extended position and the retracted position and is attached to a hand operable actuator external of the handle.

3. The device of claim 1, wherein the gripping members are spaced apart nearer to the handle and at the cotermination point are close enough together to grip a lens in place between them.

4. The device of claim 1, wherein the plunger has an upper side and a lower side and has a land on the upper side and on the lower side at the lens pushing end.

5. The device of claim 5, wherein the gripping members each have a groove facing internally and into which the land of the plunger will slide as the plunger is moved toward the extended position.

6. The device of claim 6, wherein the lands are slightly higher off the plunger than the depth of the respective grooves so that as the plunger is extended the lands will cause the grippers to separate from a secure grip on the lens to free the lens for being pushed by the plunger out of the cotermination point.

7. The device of claim 7, wherein at least one of the lands is slightly higher off the plunger than the depth of the respective groove so that as the plunger is extended the land will cause the grippers to separate from a secure grip on the lens to free the lens for being pushed by the plunger out of the cotermination point.

8. The device of claim 1, wherein at least a portion of the plunger lens pushing end is sufficiently thick that it will cause the gripping members to separate as it moves to the extended position whereby the lens is released from pressure from the gripping members.
